(19) United States
(12) Patent Application Publication
(10) Pub. No.: US 2003/0109037 A1
(43) Pub. Date: Jun. 12, 2003

(54) Methods for application of genetically-modified endogenous or exogenous stem/progenitor or their progeny for treatment of disease

(76) Inventors: Christopher Brian Reid, Alexandria, VA (US); Svetlana Pack, Gaithersburg, MD (US)

Correspondence Address: Christopher B. Reid, V 407i, 8, South Van Dorn Street, Alexandria, VA, 22304, US

(21) Appl. No.: 10/252,544

(22) Filed: Sep. 24, 2002

Related U.S. Application Data
(60) Provisional application No. 60/324,362, filed on Sep. 24, 2001.

(51) Int. Cl. C12N 5/08

(52) U.S. Cl. 435/366

(57) ABSTRACT

We propose here that endogenous stem/progenitor cells of the developing or adult nervous system be genetically modified in situ, to express therapeutically advantageous gene products. Furthermore, we propose here that endogenous or other exogenous stem cells or their progeny be genetically modified when appropriate to express advantageous gene products (and/or modified through culture techniques), and that, if exogenously derived, they be transplanted into the ventricular system of the patient nervous system, the germinal zone of the ventricular system, into postmitotic regions of the CNS or other organs.

### TECHNICAL FIELD

The field of this invention relates to 1) the in situ genetic modification in of stem/progenitors (especially of the human nervous system) for the expression of therapeutic genes. Furthermore, this invention relates to 2) the use of any human or animal derived stem/progenitor cell (especially stem cells derived from the testis) and or their progeny as well as immortalized cell lines, for the treatment of diseases in the human nervous system whether the cells be derived ultimately from the patient or from a donor source. In both aspects, this invention relates specifically to various modifications of the stem cells which would render them and/or their progeny capable of ameliorating the effects of metabolic, degenerative, mental retardative, autoimmune, immune, ischemic, microbiological, and/or toxin-mediated disease processes, age related senescence in humans and animals especially those of the human nervous system. In another aspect, this invention relates to methods of introducing genetically-modified stem cells into the patient, especially the patient's nervous system, and methods for modification of patient's endogenous stem cells in situ, in vivo.

### BACKGROUND OF THE INVENTION

Overview of Anatomy and Development

As a biological system, the mammalian nervous system represents unrivaled functional and architectural diversity. In the developing embryo, the neuroepithelium generates all of the central nervous system (CNS). Within the neuroepithelium is a population of founder neural stem cells. These stem cells divide to form progeny or daughter cells. Daughter cell fates are influenced by the immediate environment of the dividing stem/progenitor cell (including cell-cell contact, cell-matrix contact and the binding of diffusible factors to cellular receptors). On the other hand, some stem cell/progenitors appear to be indifferent to such environmental influences and demonstrate a commitment to a particular pattern of differentiation. A progenitor cell is said to be committed when it has acquired the information that ultimately dictates the phenotypes or fates of its daughter cells.

Stem/Progenitor Cells

1) Umbilical cord blood cells:During fetal deveopment, the circulation of the mother and her child normally remain separate. The blood on the fetal side of the placenta contains highly undifferentiated cells which have been shown to proliferate and differentiate under appropriate conditions to form a variety of blood cell types. Umbilical cord blood cells are becoming an increasingly important source of blood cells for bone marrow trransplant (BMT). Typically the stem cells are introduced into appropriately matched patients through injection into the patient's circulation. However, the application of these cells, with or without genetic modification, to the treatment of clinical disease by grafting into the patient CNS or other tissues for replacement of deficient gene products or supplementation of gene products is specifically covered by this invention and has not been previously attempted or published.

2) Bone marrow stem/progenitor cells permanently populate the vascular sinuses of flat and short bones over the entire lifetime of the animal, and divide to produce the relatively short-lived blood cells. This arrangement contrasts markedly with the nervous system where germinal zone cells are, for the most part, mitotically-active over a short period in the animal's lifetime, producing almost all of the adult complement of neurons, astrocytes, and oligodendrocytes prenatally. A pluripotent ciliated stem cell has nevertheless been isolated from the adult lateral ventricle of the forebrain as well the central canal of the rodent brain and spinal cord. These stem cells divide to form large, adherent masses of progeny cells (neurospheres (NS)) under appropriate conditions in culture. Interestingly, neural stem cells isolated from the embryonic or adult striatum can integrate into the bone marrow and produce a wide array of hematopoietic progeny. Conversely, genetically-marked hematopoietic progenitor cells have been found to be capable of producing neural oligodendrocytes and astrocytes when transplanted to the nervous system. These results are broadly interpreted as demonstrating that stem cells from various organ systems retain a tremendous potential to respond to unspecified morphogenetic factors and to produce both neural and non-neural cells after gaining access to the appropriate tissue environment.

3) Skin progenitor cells within the basal layer of the skin are progenitor cells which divide over the entire lifespan of the individual. Such cells are readily accessible and may be cultured by a variety of culture conditions known to the art before and during genetic modification for modification for transplantation into the nervous system or other tissues.

4) Spermatogonia and other primordial germ cells of the testis are of a special interest with regard to this application. Throughout this patent the term spermatogonia refers to all spermatogonia cells (especially spermatogonia A cells) and other primordial germ cells of the testis. Furthermore all manipulations of spermatogonia described herein should also be understood to be available for application to other stem/progenitor cells (especially, those derived from the testis, umbilical cord, blood and skin) for the same purpose.

This patent envisions isolation and preservation of spermatogonia by any method known to the art.

### DESCRIPTION

The present invention covers the genetic modification of stem/progenitor for the preparation of cells resistant to intrinsic or extrinsic disease such as immune-mediated, inflammatory, viral, bacterial, autoimmune, toxin-mediated disease, aging and/or degenerative diseases. This invention also covers the preparation of cells modified genetically to alter their responsiveness to drug therapy. This patent covers genetic modification of stem/progenitor or their progeny for the purpose of extending the life of these or other cells and genetic modification of multipotent stem/progenitor and their progeny for treatment of clinical disease, especially in the human nervous system. Finally, this patent covers the transplantation of unmodified stem cells by the same methods.

Alternatively, stem/progenitor may be modified (altered gene expression) through culture techniques to produce a desired cell line, cell type or cell class. Such techniques include exposing stem/progenitor to an exogenous agent, such as retinoic acid, or dimethylsulfoxide, promoting differentiation or modification of the stem/progenitor into the desired cell line, such as, for example, a neuronal cell line, but does not exclude the use of physiologic modifiers such as steel factor or other cytokines.

There are multiple sources of exogenous stem/progenitor cells. The present invention is directed toward the use of any genetically altered stem cell, progenitor cell, embryonic stem cell (ES cell), umbilical cord blood stem cell or immortalized cell lines, and/or their progeny for the purpose of treating disease or clinical condition, especially those of the nervous system. However bone marrow stem cells, spermatogonia, and primordial germ cells of the testis are of particular interest and are specifically covered by this invention.

A stem/progenitor cell which may be induced to differentiate into a desired cell line, cell type, or cell class. In this newly differentiated state the stem/progenitor cell (and or its progeny) are considered to be modified. The stem/progenitor cell may also be modified through genetic engineering techniques using DNA or RNA, encoding protein(s) or polypeptide(s) promoting differentiation of the stem cell into a specific cell line (for example, a neuronal cell line, a muscle cell line, or a hematopoietic cell line), cell type or cell class. The DNA or RNA may encode a transcription factor found in the particular cell lines, types (e.g. neurons, glia, muscle), or classes (e.g. neural cells, hematopoietic cells, etc.).

The term genetic modification refers to alteration of the cellular genotype by introducing natural or synthetic nucleic acids into stem/progenitor or immortalized cell lines and/or their progeny by any means known to the art. Alternatively culture conditions that induce permanent changes in gene expression patterns are considered herein to represent genetic modification. Modification of stem cells, whether they be derived from the host brain, endogenous donor sources, exogenous donor sources, or cell lines, represents a feasible approach to the treatment of certain human diseases, especially those of the human nervous system.

Genetic modifications covered by this patent would include, but are not limited to: modifications that alter the activity or amount of metabolic enzymes expressed by endogenous or exogenous stem/progenitor; modifications which alter the activity, amount, or antigenicity of cellular proteins; modifications which alter the activity or amount of proteins involved in signal transduction pathways; modifications which alter the amount or activity of structural proteins; modifications which alter the amount or activity of membrane associated proteins (structural or enzymatic); modifications which alter the activity or amount of proteins involved in DNA repair and chromosome maintenance; modifications which alter the activity or amount of proteins involved in cellular transport; modifications which alter the activity or amount of enzymes; modifications which alter the activity or amount of proteins involved in synapse formation and maintenance; modifications which alter the activity or amount of proteins involved in neurite outgrowth or axon outgrowth and formation; modifications altering the amount or activity of antioxidant producing enzymes within the cell; modifications which lead to altered post-translational modification of cellular proteins; modifications which alter the activity or amount of proteins involved in other aspects of cellular repair, and alterations which increase the lifespan of the cell (such as production of telomerase). Such proteins as those mentioned above would be encoded for by DNA or RNA derived from the human genome or other animal, plant, viral, or bacterial genomes. This invention also covers sequences designed de novo.

In the first part, this invention relates to the in situ, genetic modification of stem/progenitor cells of the nervous system for the treatment of disease. Endogenous stem cells may be modified in situ by direct injection or application of DNA or RNA vectors, including viruses, retroviruses, liposomes, etc, into the substance of the tissue or into the appropriate portion of the ventricular system. We have modified thousands of stem/progenitor cells and many thousand progeny cells in this manner. Our data shows that this manner of modifying progenitor cells results in a tremendous variety of modified cell types throughout the nervous system, and has never resulted in adverse effects. We have achieved genetic modification of stem cells in situ in multiple species.

Although it may be useful to administer neurotrophins (e.g. brain-derived neurotrophic factor (BDNF), basic fibroblast growth factor (bFGF)) prior to harvesting endogenous cells or at the time of in-situ stem cell modification, it may in most instances not be necessary. Either approach is covered by this patent.

The methods of the present invention provide an alternative to pharmacological therapy for the treatment of many diseases. Nevertheless, it may be suitable as well for modifying cells to deliver pharmaceuticals beyond the blood-brain barrier for the treatment and alleviation of diseases in the nervous system including psychiatric diseases, or to increase cellular responsivity to pharmacological therapy (including neoplastic cells).

In the second part, this invention relates to stem and progenitor cells whether they be endogenous cells in situ, or exogenous cells derived from other body regions or even other individual donors. These relatively undifferentiated, self-renewing cells (herein referred to as stem/progenitor) are very rare. Nevertheless certain sources of stem cells (such as the spermatogonia and primordial germ cell of the testis) are accessible and therefore a useful source of replacement cells in the non-fetal human.

In vitro genetic modification of exogenous cells or patient's endogenous cells are performed according to any published or unpublished method known to the art (e.g. U.S. Pat. Nos. 6,432,711, U.S. 05,593,875, U.S. 05,783,566, U.S. 5,928,944, U.S. 05,910,488, U.S. 05,824,547, etc.) or by other generally accepted means. Successfully transfected cells are identified by selection protocols involving markers such as antibiotic resistance genes. Clones from successfully transfected cells, expressing the appropriate exogenous DNA at appropriate levels, will be preserved as cell lines by cryopreservation (utilizing any appropriate method of cryopreservation known to the art).

More particularly, this invention relates to progenitor/stem/spermatogonia cells and/or their progeny (and any other stem/progenitor cell) which are modified genetically with DNA and/or RNA, and or modified through culture techniques whereby such cells become capable of differentiating into a desired primary cell line or cell class, such as neurons, glia, muscle cells etc. Throughout this patent, modified spermatogonia cells and their progeny are corporately referred to as spermatogonia or as modified spermatogonia.

It is an object of the present invention to provide modified stem/progenitor (and any other stem/progenitor cell) which are capable of differentiating uniformly into a cell line, cell type, or cell class (e.g. neural cells), not achievable by previous methods.

In accordance with an aspect of the present invention, there is provided a method of producing a desired cell line, cell type, or cell class from stem/progenitor cells. Generally, the method comprises culturing spermatogonia under conditions which promote growth of the spermatogonia at an optimal growth rate. The spermatogonia then are cultured under conditions which promote cell growth at a rate which is less than the optimal rate, and in the presence of an agent promoting differentiation of the spermatogonia into the desired cell line, cell type, or cell class (e.g. neural cells).

A growth rate which is less than the optimal growth rate, is a growth rate from about 10% to about 90% (preferably 20% to 50%) of the maximum growth rate for spermatogonia. The growth rates for spermatogonia can be determined from the doubling times of the spermatogonia

In one embodiment, when the spermatogonia cells are being cultured under conditions which promote growth of the cells at an optimal growth rate, the spermatogonia are cultured in the presence of a medium including leukemia inhibitory factor (LIF), and serum selected from the group consisting of: (i) horse serum at a concentration of from about 5% by volume to about 30% by volume; and (ii) fetal bovine serum at a concentration of from about 15% by volume to about 30% by volume. In one embodiment, the serum is horse serum at a concentration of about 10% by volume. In another embodiment, the serum is fetal bovine serum at a concentration of about 15% by volume.

In yet another embodiment, when the spermatogonia are cultured at an optimal growth rate, the spermatogonia are cultured in the absence of a feeder layer of cells.

In one embodiment, the agents(s) promoting differentiation of the spermatogonia is/are selected from the group consisting of retinoic acid and nerve growth factor, and the desired cell line, cell type, or cell class is neuronal.

In one embodiment, in addition to culturing the cells in the presence of the stimulating agent selected from the group consisting of retinoic acid and nerve growth factor, the spermatogonia are grown in the presence of a cytokine. Cytokines which may be employed include, but are not limited to, any of the neurotrophins: nerve growth factor, BDNF, GGF, etc., bFGF, EGF, PDGF, reelin, Interleukin-1, Interleukin-3, Interleukin-4, Interleukin-6, colony stimulating factors such as M-CSF, GM-CSF, and CSF-1, steel factor, and erythropoietin.

In a further embodiment, the agents(s) promoting differentiation of the spermatogonia is/are selected from the group consisting of dimethylsulfoxide and hexamethylene hisacrylamide, and the desired cell line is a muscle cell line, cell type, or cell class, such as a smooth muscle cell line, or a skeletal muscle cell line, or a cardiac muscle cell line. In one embodiment, the agents is dimethylsulfoxide. In another embodiment, the agents(s) is hexamethylene bis-acrylamide.

In one embodiment, in addition to culturing the spermatogonia in the presence of agents(s) promoting differentiation of the spermatogonia into a muscle cell line, the spermatogonia also are grown in the presence of a cytokine, examples of which are described above.

In yet another embodiment, when the spermatogonia are cultured in the presence of the agents(s) promoting differentiation of the spermatogonia into a desired cell line, cell type, or cell class, the spermatogonia also are cultured in the presence of fetal bovine serum at a concentration of about 10% by volume.

In a further embodiment, when the spermatogonia are cultured in the presence of the agents(s) promoting differentiation of the spermatogonia cells into a desired cell line, cell type, or cell class, the spermatogonia also are cultured on a three dimensional supporting structure.

Thus, the applicants submit that one may produce a desired cell line, cell type, or cell class from progenitor/stem/spermatogonia cells and/or primordial germ cells of the testis by culturing the progenitor/stem/spermatogonia cells initially under conditions which favor the growth or proliferation of such progenitor/stem/spermatogonia cells at an optimal growth rate, and then culturing the cells under conditions which decrease the growth rate of the cells and promote differentiation of the cells to a desired cell type.

In a preferred embodiment, the progenitor/stem/spermatogonia cells cultured in a standard culture medium (such as, for example, Minimal Essential Medium), which may include supplements such as, for example, glutamine, and beta.-mercaptoethanol. The medium may also include leukemia inhibitory factor (LIF), or factors with LIF activity, such as, for example, CNTF or IL-6, and horse serum. LIF, and factors with LIF activity, prevents spontaneous differentiation of the progenitor/stem/spermatogonia cells, and is removed prior to the addition of the agents(s). Horse serum promotes differentiation of the progenitor/stem/spermatogonia cells into the specific cell type after the addition of the agents(s) to the medium. After the cells have been cultured for sufficient time to permit the cells to proliferate to a desired number, the cells are washed free of LIF, and then cultured under conditions which provide for cell growth at a decreased growth rate but which also promote differentiation of the cells.

Subsequently, the cells are cultured in the presence of agents(s) promoting differentiation of the progenitor/stem/spermatogonia cells into a desired cell line, cell type, or cell class, and in the presence of fetal bovine serum at a concentration of from about 5% by volume to about 10% by volume, preferably at about 10% by volume. The presence of the fetal bovine serum at a concentration of from about 5% by volume to about 10% by volume, and of the agents(s), provides for growth or proliferation of the cells at a rate which is less than the optimal rate, while favoring the differentiation of the cells into a homogeneous desired cell type. The desired cell type is dependent upon the agents(s) promoting or stimulating the differentiation of the spermatogonia. The spermatogonia also may be cultured on a three dimensional supporting network.

For example, the spermatogonia may be placed in a culture vessel to which the cells do not adhere. Examples of non-adherent substrates include, but are not limited to, polystyrene and glass. The substrate may be untreated, or may be treated such that a negative charge is imparted to the cell culture surface. In addition, the cells may be plated in methylcellulose in culture media, or in normal culture media in hanging drops.

In order to form aggregates in hanging drops of media, cells suspended in media are spotted onto the underside of a lid of a culture dish, and the lid then is placed on the culture vessel. The cells, due to gravity, collect on the undersurface of the drop and form aggregates.

In accordance with another aspect of the present invention, there is provided a spermatogonia cell that has been modified with DNA or RNA encoding protein(s) or polypeptide(s) which promote(s) differentiation of the cell into a specific cell line, cell type, or cell class.

The DNA or RNA encoding protein(s) or polypeptide(s) promoting differentiation of the spermatogonia cell into a specific cell line, cell type, or cell class is found in the specific differentiated cell line, cell type, or cell class. Preferably, the protein or polypeptide which is present in the specific cell line, cell type, or cell class is protein(s) or polypeptide(s) which generally is not present in other types of cells.

In one embodiment, the DNA or RNA encoding protein(s) or polypeptide(s) which promote(s) differentiation of the spermatogonia cell into a specific differentiated cell line, cell type, or cell class, is present in the desired cell line, cell type, or cell class.

In one embodiment, the DNA or RNA encodes a transcription factor present in neuronal cells, and the specific cell line, cell type, or cell class is a neuronal cell line.

In another embodiment, the DNA or RNA encodes a transcription factor such as the MyoD gene, present in muscle cells, and the specific cell line is a muscle cell line.

In yet another embodiment, the DNA or RNA encodes a transcription factor present in hematopoietic cells, and the specific cell line is a hematopoietic cell line.

In yet another embodiment, the DNA or RNA encodes a transcription factor DNA or RNA encodes a transcription factor present in one cell line, cell type, or cell class but the desired cell line, cell type, or class is different from that of the transcription factor.

The DNA or RNA encoding protein(s) or polypeptide(s) promoting differentiation of the spermatogonia cell into a specific cell line may be isolated in accordance with standard genetic engineering techniques (for example, by isolating such DNA from a cDNA library of the specific cell line) and placing it into an appropriate expression vector, which then is transfected into spermatogonia.

Appropriate expression vectors are those which may be employed for transfecting DNA or RNA into eukaryotic cells. Such vectors include, but are not limited to, prokaryotic vectors such as, for example, bacterial vectors; eukaryotic vectors, such as, for example, yeast vectors and fungal vectors; and viral vectors, such as, but not limited to, lentiviral vectors, adenoviral vectors, adeno-associated viral vectors, and retroviral vectors. Examples of retroviral vectors which may be employed include, but are not limited to, those derived from Moloney Murine Leukemia Virus, Moloney Murine Sarcoma Virus, and Rous Sarcoma Virus, FIV, and HIV.

Plasmid DNA containing cDNA inserts can be electroporated into spermatogonia. Cells are transfected with a plasmid that contains sequences for an antibiotic resistance gene and stable transfectants are isolated based on antibiotic resistance. Stable transfected clones are isolated and induced with an appropriate agent, or with leukemia inhibitory factor (LIF) withdrawal alone, and scored for an increased ability to differentiate in response to these induction signals. Clones also are examined to determine if they are differentiating spontaneously in the presence of LIF.

In accordance with another aspect of the present invention, there is provided a method of producing a desired cell line, cell type, or cell class from spermatogonia. The method comprises engineering spermatogonia with DNA which encodes protein(s) or polypeptide(s) promoting differentiation of the spermatogonia into a specific cell line, type, or class. The spermatogonia then are stimulated with agents(s) promoting differentiation of the spermatogonia into the desired cell line, cell type, or cell class.

In one embodiment, the DNA which encodes protein(s) or polypeptide(s) promoting differentiation of the spermatogonia into a specific cell line is DNA encodes a transcription factor present in neuronal cells and the agents(s) is/are selected from the group consisting of retinoic acid and nerve growth factor. Alternatively, the cells also may be grown in the presence of a cytokine such as those described above.

In another embodiment, the DNA which encodes protein(s) or polypeptide(s) promoting differentiation of the spermatogonia into a specific cell line, cell type, or cell class is DNA encodes a transcription factor, such as, for example, the MyoD gene, present in muscle cells and the agents(s) is/are a bipolar agent such as dimethylsulfoxide or hexamethylene bis-acrylamide. Alternatively, the spermatogonia also may be grown in the presence of a cytokine.

The spermatogonia may be engineered with the DNA or RNA and cultured under conditions described above. For example, prior to induction, the spermatogonia are engineered with DNA which encodes protein(s) or polypeptide(s) promoting differentiation of the spermatogonia into a specific cell line, cell type, or cell class. Then, the spermatogonia may be cultured under conditions which provide for a three-dimensional arrangement of such cells.

Also, it is to be understood that, within the scope of the present invention, that the spermatogonia may be used for gene therapy purposes. The spermatogonia may be engineered with DNA encoding a desired therapeutic agent. Such engineering may be accomplished by using expression vectors such as those herein above described or others. Once the cells are engineered with DNA encoding a desired therapeutic agent, the cells then are engineered with DNA or RNA which encodes protein(s) or polypeptide(s) promoting differentiation of the spermatogonia into a specific desired cell line, cell type, or cell class, and/or stimulated with agents(s) promoting differentiation of the spermatogonia into a desired cell line, cell type, or cell class. The differentiated cells then may be administered to a host, such as a human or non-human host, as part of a gene therapy procedure.

The differentiated stem cells may be employed by means known to those skilled in the art to treat a variety of diseases or injuries. For example, stem cells which have differentiated into neuronal cells may be administered to a patient, such as, for example, by transplanting such cells into a patient, to treat diseases such as Huntington's disease, Parkinson's disease, and Alzheimer's disease. Such neuronal cells also may be employed to treat spinal cord injuries or chronic pain. Also, stem cells which have differentiated into muscle cells may be employed in treating muscular dystrophy, cardiomyopathy, congestive heart failure, and myocardial infarction, for example.

The invention will now be described with respect to the following examples; however, the scope of the present invention is not intended to be limited thereby.

### EXAMPLE 1

Undifferentiated progenitor/stem/spermatogonia are maintained in Dulbecco's modified Minimal Essential Medium (DMEM) supplemented with glutamine, beta.-mercaptoethanol, 10% (by volume) horse serum, and human recombinant Leukemia Inhibitory Factor (LIF). The LIF replaces the need for maintaining progenitor/stem/spermatogonia cells on feeder layers of cells, (which may also be employed) and is essential for maintaining progenitor/stem/spermatogonia cells in an undifferentiated state.

In order to promote the differentiation of the progenitor/stem/spermatogonia cells into neuronal cells, the progenitor/stem/spermatogonia cells are trypsinized and washed free of LIF, and placed in DMEM supplemented with 10% (by volume) fetal bovine serum (FBS). After resuspension in DMEM and 10% FBS, 1.times.10.sup.6 cells are plated in 5 ml DMEM plus 10% FBS plus 0.5 microM retinoic acid in a 60 mm Fisher brand bacteriological grade Petri dish. In such Petri dishes, progenitor/stem/spermatogonia cells cannot adhere to the dish, and instead adhere to each other, thus forming small aggregates of cells. Aggregation of cells aids in enabling proper cell differentiation. After two days, aggregates of cells are collected and resuspended in fresh DMEM plus 10% FBS plus about 0.5 microM retinoic acid, and replated in Petri dishes for an additional two days. Aggregates, now induced four days with retinoic acid, are trypsinized to form a single-cell suspension, and plated in medium on poly-D-lysine-coated coated tissue culture grade dishes. The stem cell medium is formulated with Kaighn's modified Ham's F12 as the basal medium with the following supplements added: 15 microg/ml ascorbic acid, 0.25% (by volume) calf serum, 6.25 microg/ml insulin, 6.25 microg/ml transferrin, 6.25 microg/ml selenous acid, 5.35 microg/ml linoleic acid, 30 pg/ml thyroxine (T3), 3.7 ng/ml hydrocortisone, 1. ng/ml Heparin 10 ng/ml somatostatin, 0 ng/ml Gly-His-Lys (liver cell growth factor), 0.1 microg/ml epidermal growth factor (EGF), 50 microg/ml bovine pituitary extract (BPE). This medium will provide for consistent differentiation of the stem cells into neuronal cells, and provides for survival of the neuronal cells for a period of time greater than 3 days, and selectively removes dividing non-neuronal cells from the population (U.S. Pat. No. 6,432,711). The poly-D-lysine promotes the attachment of the neuronal cells to the tissue culture plastic, and prevents detachment of the cells from the dish and the formation of floating aggregates of cells. The cells are cultured for 5 days. Upon culturing the cells in the above medium, a culture of cells in which greater than 90% of the cells are neuronal cells is obtained. Such neuronal cells, which express the neurotransmitter gamma amino butyric acid (GABA), then may be employed for the treatment of the neural degeneration disease Huntington's disease. Through genetic engineering, these cells can be directed to express dopamine (for the treatment of Parkinson's disease) or acetylcholine (for the treatment of Alzheimer's disease).

### EXAMPLE 2

Undifferentiated progenitor/stem/spermatogonia cells are maintained in supplemented Dulbecco's modified Minimal Essential Medium as described in Example 1. The progenitor/stem/spermatogonia cells then are trypsinized and washed free of LIF and placed in 1% (by volume) dimethylsulfoxide in DMEM plus 10% horse serum. Two days after the addition of dimethylsulfoxide and plating of cells in Petri dishes to form aggregates, the aggregates are collected and resuspended in fresh medium plus 1% dimethylsulfoxide. The aggregates are then plated onto multi-well untreated culture grade dishes without trypsin treatment. One aggregate is plated per well. The aggregates are cultured for 5 days. Upon culturing of the cells in multi-well dishes, cell cultures in which greater than 90% of the aggregates contain contracting muscle cells are obtained. Such cells may be used to treat cardiomyopathies, myocardial infarction, congestive heart failure, or muscular dystrophy.

### EXAMPLE 3

Progenitor/stem/spermatogonia cells can be isolated using a two-step enzymatic digestion followed by Percoll separation. Cells can then be resuspended in minimum essential medium (MEM) supplemented with bovine serum albumin to a final concentration of 10(6)/mL. In detail: Tubule fragments are accessed surgically and teased apart prior to treatment with 1 mg/ml trypsin, hyaluronidase, and collagenase, and then 1 mg/ml hyaluronidase and collagenase, in MEM containing 0.10% sodium bicarbonate, 4 mM L-glutamine, nonessential amino acids, 40 μg/ml gentamycin, 100 IU to 100 μg/ml penicillin-streptomycin, and 15 mM Hepes. Progenitor/stem/spermatogonia cells are further separated from tubule fragments by centrifugation at 30×g. After filtration through nylon filters with 77- and/or 55-μm pore sizes, cells are collected and loaded onto a discontinuous Percoll density gradient. Fractions with a purity greater than 40% progenitor/stem/spermatogonia cells are washed and resuspended to a concentration of cells equivalent to 10⁶ progenitor/stem/spermatogonia cells per milliliter. Afterwards cells will be cultured and/or stored by any cryopreservation technique known to the art.

Progenitor/stem/spermatogonia cells can be maintained in media containing 5 ng/ml human recombinant leukemia inhibitory factor instead of on feeder layers. Stable transfectants can be isolated, expanded, frozen, and then stored in liquid nitrogen. 35 independent stably transfected progenitor/stem/spermatogonia cells cell lines can be isolated.

### EXAMPLE 4

Genetic modification of progenitor/stem/spermatogonia cells may or may not require construction of genetic constructs such as DNA or RNA vectors. Genetic constructs will in most cases consist of a vector backbone, and a transactivator which regulates a promoter operably linked to a heterologous gene nucleic acid sequence. An example of a suitable vector would be a retroviral vector. Retroviruses are RNA viruses which contain an RNA genome. The gag, pol, and env genes are flanked by long terminal repeat (LTR) sequences. The 5′ and 3′ LTR sequences promote transcription and polyadenylation of mRNA's. The retroviral vector provides a regulable transactivating element, an internal ribosome reentry site (IRES), a selection marker, and a target heterologous gene operated by a regulable promoter.

Alternatively, multiple genes may under the control of multiple promoters. Finally the retroviral vector contains cis-acting sequences necessary for reverse transcription and integration. Upon infection, the RNA is reverse transcribed to DNA which integrates efficiently into the host genome. The recombinant retrovirus of this invention is genetically modified in such a way that some of the retroviral, infectious genes of the native virus have been removed and in certain instanced replaced instead with a target nucleic acid sequence for genetic modification of the cell. The transgene would typically be exogenous DNA, in its natural or altered form, from animal or plant species. In many instances the transgene would be altered to contain a signal sequence that directs the transgene's protein product to be secreted from the cell, possibly for uptake and utilization by adjacent, non-modified, host cells.

An example of a method of producing a virus whereby progenitor/stem/spermatogonia cells may be modified is as follows: “Packaging cell lines” derived from human and/or animal fobroblast cell lines are the result of transfecting or infecting normal cell lines with viral gag, pol, and env structural genes. On the other hand, packaging cell lines produce RNA devoid of the psi sequence, so that the viral particles produced from packaging cell do not contain the ga, pol, or env genes. Once vector DNA containing the psi sequence (along with the therapeutic gene) is introduced into the packaging cell, by means of transfection or infection, the packaging cell will profuce virions capable of transmitting the therapeutic RNA to the final target cell (e.g. a neuroblast).

The “infective range” of this engineered virus is determined by the packaging cell line. A number of amphotrophic packaging cell lines are availble for production of virus suitable for infecting a broad range of human cell types. These cell lines are nevertheless generally capable of encapsidating viral vectors derived from viruses which in nature usually infect different animal species. For the example vectors derived from the MMLV can nevertheless be packaged by amphotrophic cell lines.

An example protocol for producing a therapeutic viral supernatant will follow the generally, the protocol outlined below:

1. Twenty micrograms of retrovirus vector should be mixed with 2-3 micrograms of viral DNA containing the selectable marker gene (e.g. antibiotic resistance gene) by gentle tapping in 0.8-1 milliliter of Hepes buffered saline (pH=7.05) in a 1.5 ml plastic tube.

2. Seventy microliters of 2M Ca Cl2 should be added to the mixture by repeated gentle tapping.

3. When a blue precipitate first begins to appear within the tube, the product should be gently applied to a 30% confluent layer of amphotrophic packaging cells (from any number of commercial vendors). The DNA mixture should be applied only after first removing the medium from the packaging cells.

4. The packaging cells should be set to incubate for 20-30 minutes at room temperature (25 degrees Celsius) before transferring them back to an incubator at 36-38 degrees Celsius for 3.5 hours.

5. Add 3.5-4 milliliters of Hepes buffered saline containing 15% glycerol for 3 minutes then wash cell with Dulbecco's Modified Eagle's Medium (DMEM)+10% FBS x2.

6. Add back DMEM +10% FBS, and incubate cells for 20 hours at 37 degress Celsius.

7. Remove and filter medium containing therapeutic viral particles.

(Excess viral supernatant are immediately stored or concentrated and stored at −80 degrees Celsius). Supernatant may be stored with 5-8 micrograms of polybrene which may increase the efficiency of target cell infection. Otherwise polybrene may be added just before infection.

8. Stable producer lines are established by splitting packaging cell lines 1 to 20 or 1 to 40 and subsequently incubating these cells for up to 10 days (changing medium every three days) in medium containing selective drugs (e.g. certain antibiotics corresponding to transfected resistance genes).

9. After 10 days isolated colonies are picked, grown-up aliquotted and frozen for storage.

Assay of Retrovirus Infectivity/Titration are achieved by application of a defined volume of viral supernatant to a layer of confluent “test” cells such as NIH 3T3 cells plated at 20% confluence. After 2-3 cell division times (24-36 hours for NIH 3T3 cells) colonies of “test” cells incubated at 37 degrees in antibiotic-containing medium are counted. The supernatant's titer are estimated from these colony cunts by the following formula:

Colony Forming Units/ml=colonies identified×0.5(split factor)/volume of virus (ml)

The accuracy of the estimate is increased by testing large volumes of supernatant over many plates of “test” cells.

Application of the therapeutic viral supernatant to target cells may be accomplished by various means appropriate to the clinical situation.

### EXAMPLE 5

Transplantation of in vitro modified progenitor/stem/spermatogonia cells may be accomplished in the following manner: Under sterile conditions, the uterus and fetuses are visualized by ultrasound or other radiological guidance. Alternatively the uterus may be exposed surgically in order to facilitate direct identification of fetal skull landmarks. Progenitor/stem/spermatogonia cells can then be introduced by injection (using an appropriately-sized catheter or needle) or into the ventricular system, germinal zone(s), or into the substance of the nervous system. Injections may be performed in certain instances, through the mother's abdominal wall, the uterine wall and fetal membranes into the fetus. The accuracy of the injection are monitored by direct observation, ultrasound, contrast, or radiological isotope based methods, or by any other means of radiological guidance known to the art.

### EXAMPLE 6

Under appropriate sterile conditions, direct identification of fetal skull landmarks are accomplished visually as well as by physical inspection and palpation coupled with stereotaxic and radiologic guidance (see example 2). Appropriate amounts of modified progenitor/stem/spermatogonia cells can then be introduced by injection or other means into the ventricular system, germinal zones, or into the substance of the nervous system. The accuracy of the injection will be monitored by direct observation, ultrasound, or other radiological guidance.

### EXAMPLE 7

In certain, neurological diseases, such as Huntington's disease and Parkinson's disease, cells of a specific portion of the brain are selectively affected. In the case of Parkinson's disease, it is the dopaminergic cells of the substantia nigra. In such regionally-specific diseases affecting adults, radiologically-guided transplantation of modified progenitor/stem/spermatogonia cells can be undertaken under sterile conditions. Radiologic guidance will include CT and/or MRI, and take advantage contrast or isotope based techniques to monitor injected materials.

### EXAMPLE 8

In certain neurologic diseases, such as some metabolic storage disorders, cells are affected across diverse regions of the nervous system, and the greatest benefit will be achieved by introducing modified progenitor/stem/spermatogonia cells cells (or immortalized cell lines) into the tissue in large numbers in a diffuse manner. Likewise endogenous cell modification in these disorders would place a premium on modifying a large number of endogenous cells across all affected regions. In the nervous system, these diseases would be best approached by intraventricular injections (using an appropriately-sized, catheter-like device, or needle) which would allow diffuse endogenous cell modification or diffuse engraftment of in vitro modified progenitors and cell lines. However, the modified or unmodified stem cells might also be introduced directly into visceral organs, such as the liver, kidney, gut, spleen, adrenal glands, pancreas, and thymus using endoscopic guidance and any appropriately-sized, catheter-like device, allowing specific introduction and infiltration of progenitor/stem/spermatogonia cells cells into the selected organs.

### EXAMPLE 9

The ability of neuronal progenitors to produce mature blood progeny cells suggests that diseases of one organ system may be treated with genetically modified cells from a separate organ system. The treatment of blood disorders (Hereditary Spherocytosis, Sickle cell anemia, other hemoglobinopathies, etc,) for instance would involve the injection of modified progenitor/stem/spermatogonia cells directly into the circulation, by large bore intravenous needle or catheter, to “home” to the bone marrow, or directly into the bone marrow following surgical exposure of bones and introduction into the marrow space. Other diseases might be best approached by injecting modified cells directly into other organs, e.g. liver, gut, spleen, kidney, skin, lungs, etc.

### EXAMPLE 10

The term lesion is non-specific and refers to any area of cell damage or death. Modified cells may provide for replacement of neural tissue damaged in various disease such as stroke, trauma, or infection with the exciting prospect that the modified exogenous cells and their progeny would differentiate in a manner appropriate to the host environment.

### EXAMPLE 11

Data from animal studies suggests that memory function in mammals may be increased by altered expression of certain neurotransmitter receptors, such as the NMDA receptor. Thus injection of modified progenitors into the hippocampus and other memory related brain structures may be expected to ameliorate memory loss due to a variety of degenerative. We propose that clinically significant genes might be transferred to modified progenitor/stem/spermatogonia cells and their progeny in an analogous manner for the treatment of neurological and non-neurological disease according to the methods described here. Progeny cells modified in this manner demonstrate the exciting prospect of secreting by design or by “leak” pathways, gene products other molecules responsible for various clinical diseases.

### EXAMPLE 12

### In utero Injection of a Genetic Vector Such as a Retrovirus, Adenovirus, Lentivirus, or MMLV-Derived Retrovirus, for in vivo, in situ, Modification of Endogenous Cells

Modification of endogenous cells using various genetic constructs, or transplantation of in vitro modified stem/progenitor cells are accomplished by identical surgical procedures. Under sterile conditions, the uterus and fetuses are visualized by ultrasound or other radiological guidance. Alternatively the uterus may be exposed surgically in order to facilitate direct identification of fetal skull landmarks. Concentrated vectors can then be introduced by injection (using an appropriately-sized catheter or needle) or into the ventricular system, germinal zone(s), or into the substance of the nervous system. Injections may be performed in certain instances, through the mother's abdominal wall, the uterine wall and fetal membranes into the fetus. The accuracy of the injection are monitored by direct observation, ultrasound, contrast, or radiological isotope based methods, or by any other means of radiological guidance known to the art.

### EXAMPLE 13

### Postnatal Injection of a Genetic Vector such as a Retrovirus for in vivo, in situ, Modification of Endogenous Cells

Modification of endogenous cells or transplantation of in vitro modified stem/progenitor cells (or immortalized cell lines) are similarly accomplished in postnatal patients. Under appropriate sterile conditions, direct identification of fetal skull landmarks are accomplished visually as well as by physical inspection and palpation coupled with stereotaxic and radiologic guidance (see example 2). Appropriate doses of concentrated genetically-modifying vectors can then be introduced by injection or other means into the ventricular system, germinal zones, or into the substance of the nervous system. The accuracy of the injection will be monitored by direct observation, ultrasound, or other radiological guidance.

### EXAMPLE 14

### Injection of a Genetic Vector into Specific Nervous System Regions or Nuclei

In certain, neurological diseases, such as Huntington's disease and Parkinson's disease, cells of a specific portion of the brain are selectively affected. In the case of Parkinson's disease, it is the dopaminergic cells of the substantia nigra. In such regionally-specific diseases affecting adults, radiologically-guided transplantation of genetic vectors into the affected area(s) of the nervous system would be undertaken under sterile conditions. Radiologic guidance will include CT and/or MRI, and take advantage contrast or isotope based techniques to monitor injected materials.

### EXAMPLE 15

### Delivery of Modified or Unmodified Stem Cells by Injection in to the Circulatory Stream

In some instances, it may become apparent that stem cells may integrate on their own in sufficient numbers if they are injected into blood stream, either arterial, venous, or hepatic.

Examples of Specific Transgenes Covered by this Invention

In time we expect hundreds of diseases and clinical conditions to be treated and/or ameliorated by the present invention. The following represents an incomplete list of example transgenes which we will seek to have expressed by genetically-modified cells, but is no way limiting on the use of this invention: aspartoacylase in the treatment of Canavan's disease; hexosaminidase A (subunit alpha) in the treatment of Tay-Sach's disease; hypoxanthine guanine phosphoribosyltransferase in the treatment of Lesch-Nyhan syndrome; huntingtin in the treatment of Huntington's disease; beta-glucuronidase in the treatment of Sly syndrome; sphingomyelinase in the treatment of type A and type B Niemann Pick disease; the b-subunit of hexosaminidase A and hexosaminidase B in the treatment of Sandhoff's disease; alpha-galactosidase A in the treatment of Fabry's disease; the yet undiscovered mutated gene in the treatment of type C Niemann-Pick disease; the glucocerebrosidase gene in the treatment of Gaucher's disease; the presenilin genes in the treatment of Alzheimer's disease; the dopamine gene in the treatment of Parkinson's disease; The VHL gene in the treatment of Von Hippel Lindau's disease. alpha-, beta-, gamma-, and delta-subunits of hemoglobin for the treatment of sickle cell anemia and other thalassemias. These transgenes will generally represent the coding region or portions of the coding region of the normal genes.

It is to be understood, however, that the scope of the present invention is not to be limited to the specific embodiments and examples described above. The invention may be practiced other than as particularly described and still be within the scope of the accompanying claims.

What is claimed is:

1. A method for selectively producing specific cell lines, cell types or cell classes from stem/progenitor, comprising:
culturing stem/progenitor cells in a “differentiation medium” and in the presence of agents(s) promoting differentiation or modification of the stem/progenitor into desired cells selected from the group consisting of neuronal, muscle, and other cells, the stem/progenitor cell having been grown previously in a growth culture medium which differs from the “differentiation medium”, said differentiation medium promoting growth of the stem/progenitor cells at a growth rate which is from about 10% to about 90% of the growth rate of stem/progenitor in said growth culture medium, wherein when the desired cells are neuronal cells, the agents(s) is/are selected from the group consisting of retinoic acid, nerve growth factor; and wherein when the desired cells are muscle cells, the agents(s) is/are selected from the group consisting of dimethylsulfoxide and hexamethylene bis-acrylamide.

2. The process of claim 1 wherein said differentiation medium contains fetal bovine serum at a concentration of about 10% by volume.

3. The process of claim 1 wherein the agent is retinoic acid.

4. The process of claim 3 wherein, in addition to culturing the stem/progenitor cell in the presence of retinoic acid, the stem/progenitor cell are grown in the presence of a cytokine or cytokines such as steel factor.

5. The process of claim 1 wherein the agent is dimethylsulfoxide.

6. The process of claim 1 wherein the agent is hexamethylene bis-acrylamide.

7. The process of claim 1 wherein, in addition to culturing the stem/progenitor cell in the presence of agents(s) selected from the group consisting of dimethylsulfoxide and hexamethylene bis-acrylamide, the stem/progenitor cell are grown in the presence of a cytokine or cytokines.

8. A method of implanting modified, or unmodified stem/progenitor cell (endogenous or exogenous) into the nervous system or other organ to treat or prevent disease.
8a. where the stem/progenitor cells are injected into the appropriate portion(s) of the fluid-filled ventricular system.
8b. where the stem/progenitor cells are injected into the appropriate portion(s) of the germinal zones and/or regions of postmitotic differentiation (the substance) of the developing nervous system.
8c. where the stem/progenitor cells are introduced into the circulatory system (blood).

9. The process of claim 1 wherein “stem/progenitor cell” are replaced by stem cells derived from umbilical cord blood, bone marrow, brain, testis or skin cells.

10. A method of modifying in situ endogenous stem/progenitor cells of the developing mammalian nervous system, to treat or prevent disease.
10a. where the modifying genetic construct(s) are injected into the appropriate portion(s) of the fluid-filled ventricular system.
10b. where the modifying genetic construct(s) injected into the appropriate portion(s) of the germinal zones and/or regions of postmitotic differentiation (the substance) of the developing nervous system.
10c. where the modifying genetic construct(s) are introduced into the circulatory system (blood).
